(19) United States
(12) Patent Application Publication
(10) Pub. No.: US 2005/0282163 A1
(43) Pub. Date: Dec. 22, 2005

(54) Polymorphisms in elastin, fibrillin and related genes as predisposing to restenosis and to a therosclerosis

(75) Inventor: Stephen E. Epstein, Rockville, MD (US)

Correspondence Address: HELLER EHRMAN WHITE & MCAULIFFE LLP, 1717 RHODE ISLAND AVE, NW, WASHINGTON, DC, 20036-3001, US

(73) Assignee: Medstar Research Institute Washington Hospital Center, Washington, DC (US)

(21) Appl. No.: 10/502,984

(22) PCT Filed: Feb. 3, 2003
(86) PCT No.: PCT/US03/03001

§ 371 Date: Jun. 6, 2005

(51) Int. Cl. C12Q 1/68; A61K 39/395; A61K 48/00

(52) U.S. Cl. 435/6; 514/44; 424/130.1

(57) ABSTRACT

Methods are provided for assessing the risk of developing restenosis in an individual, by detecting the presence of biologically important polymorphisms in genes involved in the formation of the elastin fiber network. In particular, detection of polymorphorphisms in the elastin, fibrillin, fibrillin-1, fibrillin-2, lysyl oxidase, microfibril-associated glycoprotein, biglycan, osteopontin, and/or decorin genes allows the rapid and objective prediction of the risk of developing restenosis. Methods for treating restenosis and for reducing its recurrence also are provided.

### I. BACKGROUND OF THE INVENTION

Coronary artery disease is a disease that is endemic in Western society. In this disease the arteries that supply blood to the heart muscle become narrowed by deposits of fatty, fibrotic, or calcified material on the inside of the artery. The build up of these deposits is called atherosclerosis. Atherosclerosis reduces the blood flow to the heart, which starves the heart muscle of oxygen, leading to either/or angina pectoris (chest pain), myocardial infarction (heart attack), and congestive heart failure.

One common treatment to clear arteries blocked by atherosclerosis is balloon angioplasty, more formally referred to as percutaneous transluminal coronary angioplasty (PTCA). This treatment involves opening up a blocked artery by inserting and inflating a small balloon, which compresses and rearranges the blocking plaque against the arterial wall. After deflation and removal of the balloon, the arterial lumen is enlarged, thereby improving blood flow. About one million angioplasty procedures are performed each year.

In a significant number of angioplasty patients the treated artery narrows again within six months of the procedure in a process called restenosis. Restenosis begins soon after angioplasty, wherein the increased size of the vascular lumen (the open channel inside the artery) becomes gradually occluded by the proliferation of smooth muscle cells. Approximately 20 to 30% of all angioplasty patients experience restenosis to the extent that they must undergo repeated angioplasty or even coronary bypass surgery.

Restenosis has a complex pathology, triggered by the stretch-induced injury of the vessel walls during balloon inflation. The injury involves several components of the vessel wall, including the elastin fibers. The injury stimulates smooth muscle cell migration and proliferation, and thereby leads to neointimal accumulation (which constitutes the restenotic lesion). Additional processes contributing to restenosis include inflammation and accumulation of extracellular matrix. Remodeling of the vessel wall, leading to narrowing of the vessel, is a critically important component of restenosis. However, this is totally eliminated by the emplacement of a stent at the site of angioplasty, which prevents the vessel from remodeling. Stenting has become almost routine, being performed in many centers in over 70% of all angioplasty procedures. Restenosis also occurs in the arteries supplying the legs when these vessels are narrowed by atherosclerosis and are treated by angioplasty.

Currently, restenosis is diagnosed by visualizing the narrowed vessel through the injection of radioopaque dye into the vessel being examined and performing a cineangiogram (angiography). Typically, restenosis is diagnosed if the post-angioplasty diameter of the vessel narrows to less than 50%, a process, when it occurs, that usually within 2-6 months following the procedure.

While several factors appear to be related to the occurrence of restenosis, including diabetes, the number of times the procedure has been performed, or the placement of a stent in the vessel, there presently are no reliable predictive indicators for the large majority of patients as to whether or not a given patient is at high risk for the development of restenosis. If a reliable risk profile were available, it would importantly influence how the patient were treated. Some patients deemed to be at very high risk for restenosis might be offered bypass surgery. Others might forego angioplasty and treated very aggressively with medical management. In still others brachytherapy (intravascular radiation) might be added to the usual angioplasty, a procedure normally reserved for patients who are now identified as being at high risk of restenosis using a rather blunt assessment—they already have had multiple episodes of restenosis. It is apparent, therefore, that new and improved methods for detecting and treating restenosis are greatly to be desired.

Finally, it is commonly appreciated that restenosis shares, with atherosclerosis, many common and overlapping processes and mechanisms. One of the key differences in these two conditions is the speed with which functionally important narrowing of the involved artery develops. Hence, restenosis can be used as an efficient model to understand many of the mechanisms responsible for atherosclerosis, and the genetic factors contributing to restenosis also probably contribute to atherosclerosis.

### II. SUMMARY OF THE INVENTION

It is therefore an object of this invention to provide methods that rapidly and objectively predict the risk of the development of restenosis.

It is a further object of this invention to provide more efficacious means of treating restenosis and to reduce its recurrence.

In accordance with these goals the inventor has identified a set of genes that encode proteins present in the extracellular matrix of the artery, and which are injured during the acute injury imposed on the vessel during angioplasty, and during the chronic injury imposed on the vessel during the process of atherogenesis. The genes identified are those involved in the formation of the elastin fiber network that is present in vessel walls. These genes consist of, but are not limited to, the genes encoding elastin/tropoelastin, fibrillin-1 and fibrillin-2, lysyl oxidase, and microfibril-associated glycoprotein (MAGP), and the genes encoding the dermatan sulphate proteoglycans, biglycan, osteopontin (Eta-1), and decorin. The protein products of these genes contribute to the formation of elastin fibers, briefly, as follows; tropoelastin is secreted by cells, and is laid down along a cytoskeletal matrix support structure composed of microfibrils, which are in turn formed mainly by fibrillin-1 and 2. Through complex biochemical processes the soluble tropoelastin transforms into the insoluble elastin. This is in part accomplished by the enzyme lysyl oxidase, which has been shown to incorporate soluble tropoelastin into insoluble elastin fibers with the formation of elastin crosslinks. The dermatan sulphate proteoglycans, biglycan, and decorin, have been found to bind to tropoelastin, but their exact role in the function of elastic fibers is not known. Osteopontin is a constitutive component of normal elastic fibers in human skin and aorta (L. J. van't Veer et al., Nature 415:530-536, Jan. 31, 2002). It is present in human coronary artherosclerotic plaques, but not in non-diseased vessels.

The key to an understanding of the rationale of this invention is the recognition that elastin fibers not only forms a structural supporting network for the vessel, but, when intact, elastin induces the quiescent phenotype of SMCs residing within the vessel media. When elastin is disrupted or absent, SMCs no longer retain the quiescent phenotype—instead they proliferate and migrate to form neointimal hyperplasia, the key process involved in both restenosis and atherosclerosis.

Genetic mutations of elastin have been found to lead to supravalvular aortic stenosis, characterized by narrowing (stenosis) of the aorta as it emerges from the heart just distal to the aortic valve. The stenosis is caused by migration and proliferation of SMCs located in the media of the aorta—this causes neointimal hyperplasia and ultimately narrowing of the aortic lumen such that obstruction to blood flow develops (supravalvular aortic stenosis). Genetic mutations of fibrillin proteins also can lead to vascular diseases. It is the concept of this invention that polymorphisms of the genes encoding proteins involved in the formation of the elastin fiber network, which alter the functional integrity of elastin, will predispose to SMC migration and proliferation, and thereby to obstruction of the coronary, cerebral, or peripheral arteries. These processes could occur in the context of angioplasty and predispose to restenosis, or chronic vascular injury, predisposing to atherosclerosis.

The inventor recognizes that since the differential expression of these genes is involved in the healing response to vascular injury, whereby intact elastin fibers are necessary for SMCs to resume their quiescent phenotype, changes in the degree of expression, or in the length of time during which elastin and genes contributing to the functional integrity of elastin (as noted above) are differentially expressed, could lead to abnormal patterns of healing. Analogous to a keloid scar, in which a genetic precondition leads to excessive fibrous tissue developing on the skin in response to cutaneous injury, in the context of injury to the vessel wall (either acute as in restenosis or chronic as in atherosclerosis) the excessive healing response would contribute to the development of restenosis or atherosclerosis.

The inventor further recognizes that changes in the degree of gene expression, or in the length of time during which the genes are expressed, can be caused by polymorphisms in the gene or in the regulatory components of the gene. Such polymorphisms, conveying an increased risk of disease development, have already been identified for several genes associated with several diseases. This invention, therefore, identifies the genes encoding elastin/tropoelastin, fibrillin-1 and fibrillin-2, lysyl oxidase, and microfibril-associated glycoprotein (MAGP), and the genes encoding the dermatan sulphate proteoglycans, biglycan, osteopontin, and decorin as genes in which polymorphisms can convey susceptibility to the development of restenosis or atherosclerosis. The invention is not limited to these specific genes, as it involves all genes whose protein products influence the integrity of elastin fibers: Subsequent reference, therefore, to prediction of restenosis (or atherosclerosis-see below), relate to polymorphisms of the genes identified by this invention, or of their regulatory units.

In view of the importance that the identified genes may play in the etiology of restenosis or atherosclerosis, the inventor recognizes that an ability to manipulate the expression of those genes may be efficacious in the treatment of restenosis or atherosclerosis. Methods to treat may include gene therapy to increase the expression of genes down-regulated during the disease. Treatment may also include methods to decrease the expression of genes up-regulated during restenosis/atherosclerosis. Treatment to decrease gene expression may include, but is not limited to, the expression of anti-sense mRNA, triplex formation or inhibition by co-expression.

The inventor further recognizes that identification of the genes encoding elastin/tropoelastin, fibrillin-1 and fibrillin-2, lysyl-oxidase, and microfibril-associated glycoprotein (MAGP), and the genes encoding the dermatan sulphate proteoglycans, biglycan and decorin (and any other genes whose protein products influence the functional integrity of elastin), which influence the development of restenosis/atherosclerosis, makes possible an identification of proteins that may effect the development of restenosis/atherosclerosis. Identification of such proteins makes possible the use of methods to affect their expression or alter their metabolism. Methods to alter the effect of expressed proteins include, but are not limited to, the use of specific antibodies or antibody fragments that bind the identified proteins, specific receptors that bind the identified protein, or other ligands or small molecules that inhibit the identified protein from affecting its physiological target and exerting its metabolic and biologic effects. In addition, those proteins that are down-regulated during the course of restenosis may be supplemented exogenously to ameliorate their decreased synthesis.

The inventor realizes that identification of the genes encoding elastin/tropoelastin, fibrillin-1 and fibrillin-2, lysyl oxidase, and microfibril-associated glycoprotein (MAGP), and the genes encoding the dermatan sulphate proteoglycans, biglycan, osteopontin, and decorin (and any other genes whose protein products influence the functional integrity of elastin), which influence the development of restenosis/atherosclerosis, makes possible the prophylactic use of methods to affect gene expression or protein function, and such methods may be used to treat individuals at risk for the development of restenosis/atherosclerosis.

The inventor further recognizes that different polymorphisms of the genes encoding elastin/tropoelastin, fibrillin-1 and fibrillin-2, lysyl oxidase, and microfibril-associated glycoprotein (MAGP), and the genes encoding the dermatan sulphate proteoglycans, biglycan, osteopontin, and decorin (and any other genes whose protein products influence the functional integrity of elastin, which influence the development of restenosis/atherosclerosis, may play a role in the development of restenosis/atherosclerosis in different patients. The inventor therefore recognizes that this invention makes possible an identification of specific abnormalities that are characteristic of a specific patient. The inventor recognizes that this would allow for greater specificity of treatment. A regime that may be efficacious in one patient with a specific polymorphism profile may not be effective in a second patient with a different polymorphism profile. Such a profiling would also allow treatment to be individualized so that unnecessary side effects of a treatment strategy that would not be effective for a specific patient can be avoided.

Finally, the inventor realizes that restenosis shares, with atherosclerosis, many common and overlapping processes and mechanisms. One of the key differences in these two conditions is the speed at which functionally important narrowing of the involved artery develops. Hence, restenosis can be used as an efficient model to understand many of the mechanisms responsible for atherosclerosis. The inventor therefore realizes that.

The inventor has discovered, using transcriptional profiling (DNA arrays) in their rat carotid injury model that the genes encoding elastin, fibrillin-1, and fibrillin-2 are differentially upregulated following injury, indicating that these genes the elastin or fibrillin genes, or any of the other genes contributing to the functional integrity of elastin as noted above, are important in the response to vascular injury. The invention therefore predicts that abnormal transcriptional regulation of these genes, which could occur as a result of polymorphisms in the coding or regulatory regions of the genes, will predispose to the development of restenosis/atherosclerosis.

It follows that the inventor has discovered that identification of polymorphisms in the elastin gene or fibrillin genes, or any of the other genes contributing to the functional integrity of elastin as noted above, may be predictive of restenosis/atherosclerosis. As such, identification of these polymorphisms can help predict the patient at risk, and thereby identify patients who should be treated more aggressively to prevent the development of restenosis/atherosclerosis.

Further, the inventor has discovered that the expression of the elastin or fibrillin genes (or any of the other genes contributing to the functional integrity of elastin as noted above) are altered during the healing response to acute vascular injury, and therefore during the course of atherosclerosis.

In view of the importance the identified genes may play in the etiology of restenosis/atherosclerosis, the inventor recognizes that an ability to manipulate the expression of those genes may be efficacious in the treatment of restenosis/atherosclerosis. Methods to treat atherosclerosis may include gene therapy to increase the expression of genes down-regulated during the healing response to acute vascular injury and therefore during atherosclerosis. Treatment may also include methods to decrease the expression of genes up-regulated during the healing response to acute vascular injury and therefore during atherosclerosis. Treatment to decrease gene expression may include, but is not limited to, the expression of anti-sense mRNA, triplex formation or inhibition by co-expression.

The inventor further recognizes that identification that the elastin and fibrillin genes (and any of the other genes contributing to the functional integrity of elastin as noted above) are involved in the healing response to acute vascular injury and therefore in the development of restenosis/atherosclerosis makes possible an identification of proteins that may effect the development of atherosclerosis. Identification of such proteins makes possible the use of methods to affect their expression or alter their metabolism. Methods to alter the effect of expressed proteins include, but are not limited to, the use of specific antibodies or antibody fragments that bind the identified proteins, specific receptors that bind the identified protein, or other ligands or small molecules that inhibit the identified protein from affecting its physiological target and exerting its metabolic and biologic effects. In addition, those proteins that are down-regulated during the healing response to acute vascular injury and therefore during the course of restenosis/atherosclerosis may be supplemented exogenously to ameliorate their decreased synthesis.

The inventor realizes that the identification of the elastin and fibrillin genes (and any of the other genes contributing to the functional integrity of elastin as noted above) as being involved in the healing response to acute vascular injury and therefore in the development of restenosis/atherosclerosis makes possible the prophylactic use of methods to affect gene expression or protein function, and such methods may be used to treat individuals at risk for the development of atherosclerosis.

The inventor further recognizes that different polymorphisms may play a role in the development of restenosis/atherosclerosis in different patients. The inventor therefore recognizes that this invention makes possible an identification of specific abnormalities that are characteristic of a specific patient. The inventor recognizes that this would allow for greater specificity of treatment. A regime that may be efficacious in one patient with a specific polymorphism profile may not be effective in a second patient with a different polymorphism profile. Such a profiling would also allow treatment to be individualized so that unnecessary side effects of a treatment strategy that would not be effective for a specific patient can be avoided.

Other objects, features and advantages of the present invention will become apparent from the following detailed description. It should be understood, however, that the detailed description, while indicating preferred embodiments of the invention, are given by way of illustration only, since various changes and modifications within the spirit and scope of the invention will become apparent to those skilled in the art from this detailed description.

### III. BRIEF DESCRIPTION OF THE DRAWINGS

FIG. 1 shows the differential expression of the tropoelastin gene and the fibrillin-1 gene following acute vascular injury of the rat carotid artery.

### IV. DETAILED DESCRIPTION OF THE INVENTION

The invention provides new and improved methods for prediction, prevention, and treatment of restenosis and of atherosclerosis. The elastin and fibrillin genes (and any of the other genes contributing to the functional integrity of elastin as noted above) have been identified as having altered expression levels during the healing response to acute vascular injury, and therefore during restenosis and during atherosclerosis.

Because differential expression of these genes are involved in the healing response to vascular injury, changes in the degree of their expression, or in the length of time during which they are expressed, would lead to abnormal patterns of healing. In the context of injury to the vessel wall (either acute as in restenosis or chronic as in atherosclerosis), the excessive healing response would contribute to the development of either restenosis or atherosclerosis. Changes in the degree of gene expression, or in the length of time during which the genes are differentially expressed, are caused by polymorphisms in the gene or in the regulatory components of the gene. This invention, therefore, identifies the elastin gene, or any of the other genes contributing to the functional integrity of elastin as noted above, in which polymorphisms can convey susceptibility to the development of either restenosis or atherosclerosis.

The identification of the elastin and fibrillin genes (and any of the other genes contributing to the functional integrity of elastin as noted above) as being involved in the healing response to acute vascular injury allows for identification of these genes whose changed degree or duration of expression caused by polymorphisms of the gene, as targets to identify genetic abnormalities that convey altered risk of restenosis or atherosclerosis. Identification of such polymorphisms associated with increased risk allows prediction of the risk for restenosis development in patients prior to the performance of the angioplasty procedure, This pre-procedure risk prediction will importantly influence how the patient is treated. Some patients deemed to be at very high risk for restenosis might be offered bypass surgery. Others might forego angioplasty and be treated aggressively with medical management. In still others brachytherapy (intravascular radiation) might be added to the usual angioplasty, a procedure normally reserved for patients who are now identified as being at high risk of restenosis using a rather blunt assessment—they already have had multiple episodes of restenosis. It is apparent, therefore, this invention offers a new and improved method for predicting risk of restenosis.

Moreover, identification of the elastin and fibrillin genes (and any of the other genes contributing to the functional integrity of elastin as noted above) as genes that are activated during the healing response to acute vascular injury provides new methods for preventing, ameliorating, or treating the disease by targeted inhibition of the expression of a suitable set or subset of those genes.

Furthermore, restenosis shares, with atherosclerosis, many common and overlapping processes and mechanisms. Therefore, many of the genes differentially expressed during the healing response to acute vascular injury are the same genes differentially expressed during chronic vascular injury leading to atherosclerosis. The invention therefore also allows risk profiling of individuals for the development of atherosclerosis prior to the actual development of clinically significant atherosclerosis; i.e. prior to the development of detectable or significant narrowing of the relevant cardiac artery or peripheral arteries. This information therefore allows prophylactic intervention to prevent atherosclerosis, and prompt detection to allow delay or amelioration of the disease process.

The invention also allows the identification of the elastin gene, or any of the other genes contributing to the functional integrity of elastin as noted above, as genes to be analyzed for polymorphisms that predispose to atherosclerosis risk. Because different polymorphisms play a role in the development of atherosclerosis in different patients, the invention allows identification of specific abnormalities that are characteristic of a specific patient. The invention therefore allows for greater specificity of treatment. A regime that may be efficacious in one patient with a specific polymorphism profile may not be effective in a second patient with a different polymorphism profile. Such a profiling also allows treatment to be individualized so that unnecessary side effects of a treatment strategy that would not be effective for a specific patient can be avoided.

Elucidation of Changes in Gene Expression in Restenosis/atherosclerosis

The rat is a widely accepted model for the human for vascular studies, and results obtained in the rat are considered highly predictive of results in humans. Accordingly, it is expected that, as found in the rat, there will be differences in gene expression of the elastin and fibrillin genes, (and some of the other genes contributing to the functional integrity of elastin as noted above), in humans during the healing response to acute or chronic vascular injury. Exaggerated changes in the degree of expression in these genes, or in the length of time during which the genes are differentially expressed, will predispose to restenosis/atherosclerosis. Because such exaggerated changes are usually caused by polymorphisms in the gene or in the regulatory components of the gene, the rat genes identified as being differentially regulated during the healing response to acute vascular injury will be the homologous human genes in which such polymorphisms will be found to convey susceptibility to restenosis. Because restenosis shares many of the processes and mechanisms as atherosclerosis, and since both result from vascular injury, then the genes identified in the rat model of the healing response to acute vascular injury will also be the genes whose abnormal expression will predisponse to atherosclerosis. That this is the case is suggested by studies demonstrated that mutations of the elastin gene leading to lack of expression of elastin leads to SMC migration and proliferation, and to supravalvular aortic stenosis. Moreover, mutations in the fibrillin genes have also been shown to produce vascular disease.

The specific abnormalities will be determined by identifying polymorphisms of the elastin or fibrillin genes (or any of the other genes contributing to the functional integrity of elastin as noted above) that are associated with restenosis/atherosclerosis. Such polymorphisms will also serve indicate which gene should serve as the target for therapeutic interventions—those genes whose polymorphisms cause an upregulation of gene transcription can be targeted by therapy designed to decrease gene expression or function of the proteins encoded by these genes; those genes in which identified polymorphisms cause down-regulation of transcription can be targeted by therapy designed to increase gene expression or function of the proteins encoded by these genes.

Once functionally relevant polymorphisms are associated with restenosis/atherosclerosis, it will be possible to predict the risk of restenosis/atherosclerosis developing by ascertaining whether such polymorphisms are present in a given patient.

By assaying the presence of polymorphisms that influence expression of the elastin or fibrillin genes (or any of the other genes contributing to the functional integrity of elastin as noted above) it is possible to predict the risk of restenosis development prior to performing the angioplasty procedure, and predict the risk of atherosclerosis development prior to the development of clinically detectable atherosclerosis. Such early prediction provides the clinician with opportunities to slow or halt the restenosis or atherosclerosis processes. Moreover, the invention provides new compositions that can be used to inhibit, slow, or prevent restenosis and atherosclerosis.

Dysregulation of the Elastin or Fibrillin Genes (or Any of the Other Genes Contributing to the Functional Integrity of Elastin as Noted Above)

The identification of gene polymorphisms that lead to biologically important exaggerated changes in the expression of the elastin or fibrillin genes (or any of the other genes contributing to the functional integrity of elastin as noted above), can be measured directly in patient samples. These samples are DNA that is most conveniently obtained from peripheral blood. However, other methods for measuring changes in gene expression are well known in the art. For example, levels of proteins can be measured in tissue sample isolates using quantitative immunoassays such as the ELISA. Kits for measuring levels of many proteins using ELISA methods are commercially available from suppliers such as R&D Systems (Minneapolis, Minn.) and ELISA methods also can be developed using well known techniques. See for example Antibodies: A Laboratory Manual (Harlow and Lane Eds. Cold Spring Harbor Press). Antibodies for use in such ELISA methods either are commercially available or may be prepared using well known methods.

Other methods of quantitative analysis of multiple proteins include, for example, proteomics technologies such as isotope coded affinity tag reagents, MALDI TOF/TOF tandem mass spectrometry, and 2D-gel/mass spectrometry technologies. These technologies are commercially available from, for example, Large Scale Proteomics Inc. (Germantown Md.) and Oxford Glycosystems (Oxford UK).

Alternatively, quantitative mRNA amplification methods, such as quantitative RT-PCR, can be used to measure changes in gene expression at the message level. Systems for carrying out these methods also are commercially available, for example the TaqMan system (Roche Molecular System, Alameda, CA) and the Light Cycler system (Roche Diagnostics, Indianapolis, Ind.). Methods for devising appropriate primers for use in RT-PCR and related methods are well known in the art. In particular, a number of software packages are commercially available for devising PCR primer sequences.

Nucleic acid arrays offer are a particularly attractive method for studying the expression of multiple genes. In particular, arrays provide a method of simultaneously assaying expression of a large number of genes. Such methods are now well known in the art and commercial systems are available from, for example, Affymetrix (Santa Clara, Calif.), Incyte (Palo Alto, Calif.), Research Genetics (Huntsville, Ala.) and Agilent (Palo Alto, Calif.). See also U.S. Pat. Nos. 5,445,934, 5,700,637, 6,080,585, 6,261,776 which are hereby incorporated by reference in their entirety.

The inventor further recognizes that changes in the degree of gene expression, or in the length of time during which the genes are differentially expressed, can be caused by polymorphisms in the gene or in the regulatory components of the gene. Such polymorphisms, conveying an increased risk of disease development, have already been identified for several genes associated with several diseases. This invention, therefore, identifies the elastin or fibrillin genes (or any of the other genes contributing to the functional integrity of elastin as noted above) in which polymorphisms can convey susceptibility to the development of restenosis/atherosclerosis. Subsequent reference, therefore, to prediction of restenosis (or atherosclerosis-see below), relate to polymorphisms of these genes, or of their regulatory units.

Tissues Sampled to Determine the Presence of Polymorphisms that Cause Biologically Important Alterations in Relevant Gene Expression

Although any sample containing DNA would be appropriate, the simplest tissue to sample is peripheral venous or arterial blood.

Methods of Studying Polymorphisms of the Gene Listed in Table 1

Polymorphisms can be identified by several methods including sequencing, short tandem repeat association studies, single nucleotide polymorphism association studies, etc. These methods are well-known to anyone well-versed in the art.

Gene expression can also be studied at the protein level. While each cell nucleus carries a complete set of genes only those genes expressed in each cell are transcribed into mRNA which is then translated into proteins. Consequently, gene expression is tissue or even cell specific. Generally, it is thought that the greater the number of RNA molecules transcribed the greater the number of protein molecules translated from them and, accordingly, the results obtained using protein analysis should be the same, at least in terms of relative changes in levels of gene expression. An analysis of gene expression may therefore be directed at the quantity of a particular mRNA transcript or the amount of protein translated from it. However, although gene polymorphisms are detected reliably with tissue derived from any source, including peripheral blood, assay of the mRNA or protein encoded by the elastin and fibrillin genes (and any of the other genes contributing to the functional integrity of elastin as noted above) to determine relevant changes in the level of gene expression is critically dependent on tissue sampled. While some idea of altered gene expression occurring at the site of developing restenosis or of atherosclerosis can be obtained from sampling and testing peripheral blood, much more reliable estimates of altered gene expression would be obtained from sampling the actually artery developing restenosis or of atherosclerosis.

RNA Expression

Methods of isolating RNA from tissue are well known in the art. See, for example, Sambrook et al. Molecular Cloning: A Laboratory Manual (Third Edition) Cold Spring Harbor Press, 2001. Commercial reagents also are available for isolating RNA.

Briefly, for example, cells or tissue are lysed and the lysed cells centrifuged to remove the nuclear pellet. The supernatant is then recovered and the nucleic acid extracted using phenol/chloroform extraction followed by ethanol precipitation. This provides total RNA, which can be quantified by measurement of optical density at 260-280 nM.

mRNA can be isolated from total RNA by exploiting the “PolyA” tail of mRNA by use of several commercially available kits. QIAGEN mRNA Midi kit (Cat. No. 70042); Promega PolyATtract®. mRNA Isolation Systems (Cat. No. Z5200). The QIAGEN kit provides a spin column using Oligotex Resin designed for the isolation of poly A mRNA and yields essentially pure mRNA from total RNA within 30 minutes. The Promega system uses a biotinylated oligo dT probe to hybridize to the mRNA poly A tail and requires about 45 minutes to isolate pure mRNA.

Genetic Microarray Analysis

Microarray technology is an extremely powerful method for assaying the expression of multiple genes in a single sample of mRNA. For example, Gene Chip® technology commercially available from Affyinetrix Inc. (Santa Clara, Calif.) uses a chip that is that is plated with probes for over thousands of known genes and expressed sequence tags (ESTs). Biotinylated cRNA (linearly amplified RNA) is prepared and hybridized to the probes on the chip. Complementary sequences are then visualized and the intensity of the signal is commensurate with the number of copies of mRNA expressed by the gene.

Quantitative PCR

Quantitative PCR (qPCR) employs the co-amplification of a target sequence with serial dilutions of a reference template. By interpolating the product of the target amplification with that a curve derived from the reference dilutions an estimate of the concentration of the target sequence may be made. Quantitative reverse transcription PCR (RTPCR) may be carried out on mRNA using kits and methods that are commercially available from, for example, Applied BioSystems (Foster City, Calif.) and Stratagene (La Jolla, Calif.) See also Kochanowsi, Quantitative PCR Protocols” Humana Press, 1999. For example, total RNA may be reverse transcribed using random hexamers and the TaqMan Reverse Transcription Reagents Kit (Perkin Elmer) following the manufacturer's protocols. The cDNA is amplified using TaqMan PCR master mix containing AmpErase UNG dNTP, AmpliTaq Gold, primers and TaqMan probe according to the manufacture's protocols. The TaqMan probe is target-gene sequence specific and is labeled with a fluorescent reporter (FAM) at the 5′ end and a quencher (e.g. TAMRA) at the 3′ end. Standard curves for both endogenous control and the target gene may be constructed and the comparison of the ration of CT (threshold cycle number) of target gene to control in treated and untreated cells is determined. This technique has been widely used to characterize gene expression.

Protein Expression

Gene expression may also be studied at the protein level. Target tissue is first isolated and then total protein is extracted by well known methods. Quantitative analysis is achieved, for example, using ELISA methods employing a pair of antibodies specific to the target protein.

A subset of the proteins encoded by genes related to the functional integrity of elastin are soluble or secreted. In such instances the proteins may be found in the blood, piasma or lymph and an analysis of those proteins may be afforded by any of those methods described for the analysis of proteins in such tissues. This provides a minimally invasive means of obtaining patient samples for estimate of risk of developing restenosis or of atherosclerosis.

Treatment of Restenosis

The identification of the set of genes encompassing the elastin or fibrillin genes (or any of the other genes contributing to the functional integrity of elastin as noted above) having altered expression during the healing response to vascular injury, provides new opportunities to treat restenosis or atherosclerosis. Identification of genes up-regulated during the healing response to vascular injury affords the ability to use methods to negatively affect their transcription or translation. Similarly, the identification of genes that are down-regulated during the healing response to vascular injury affords the ability to positively affect their expression. Finally, the identification of the proteins encoded by these genes allows for the use of appropriate methods to ameliorate or potentiate the protein activities, which thereby could influence the development of restenosis or atherosclerosis.

Methods of Enhancing Gene Expression

For the elastin or fibrillin genes (or any of the other genes contributing to the functional integrity of elastin as noted above) that exhibit decreased expression during the healing response to vascular injury, it is possible to ameliorate or prevent restenosis or atherosclerosis by enhancing expression of one or more of these genes. Gene transcription may be deliberately modified in a number of ways. For example, exogenous copies of a gene may be inserted into the genome of cells in vascular tissue by genomic transduction via homologous recombination. While expression by genomic transduction is relatively stable it also is of low efficiency. An alternative method is transient transduction where the gene is inserted within a vector allowing for its transcription independent of the genomic allele making use of a vector specific promoter. Yet another method is transfection with naked DNA. However, this method generally results in very low expression.

Methods of Inhibiting of Gene Expression

The present invention also affords an ability to negatively affect the expression of the elastin or fibrillin genes (or any of the other genes contributing to the functional integrity of elastin as noted above) that are up-regulated during the healing response to vascular injury. Methods for down regulating genes are well known. It has been shown that antisense RNA introduced into a cell will bind to a complementary mRNA and thus inhibit the translation of that molecule. In a similar manner, antisense single stranded cDNA may be introduced into a cell with the same result. Further, co-suppression of genes by homologous transgenes may be effected because the ectopically integrated sequences impair the expression of the endogenous genes (Cogoni et al. Antonie van Leeuwenhoek, 1994; 65(3):205-9), and may also result in the transcription of antisense RNA (Hamada w and Spanu PD; Mol. Gen Genet 1998). Methods of using short interfering RNA (RNAi) to specifically inhibit gene expression in eukaryotic cells have recently been described. See Tuschl et al., Nature 411:494-498 (2001).

In addition, stable triple-helical structures can be formed by bonding of oligodeoxyribonucleotides (ODNs) to polypurine tracts of double stranded DNA. (See, for example, Rininsland, Proc. Nat'l Acad. Sci. USA 94:5854-5859 (1997). Triplex formation can inhibit DNA replication by inhibition of transcription of elongation and is a very stable molecule.

Methods to Inhibit the Activity of Specific Proteins

When a specific protein has been implicated in the restenotic or atherosclerotic pathway its activity can be altered by several methods. First, specific antibodies may be used to bind the protein thereby blocking its activity. Such antibodies may be obtained through the use of conventional hybridoma technology or may be isolated from libraries commercially available from Dyax (Cambridge, Mass.), MorphoSys (Martinsried, Germany), Biosite (San Diego, Calif.) and Cambridge Antibody Technology (Cambridge, UK). In addition, proteins usually exert their cellular effects by ligating to cellular receptors. Identification of the receptors to which proteins, which are implicated by the current invention as contributing to restenosis or atherosclerosis, bind will allow the design of specific ligand antagonists that block pathways mediating the effects leading to the development of restenosis or atherosclerosis.

The identification of genes that are down regulated during the healing response to acute vascular injury leads to the ability to identify their protein products. Down-regulated proteins may then be supplemented, thereby ameliorating the effect of their decreased synthesis.

The methods of the present invention may be used prophylactically to prevent the development of restenosis or atherosclerosis in at risk individuals.

The present invention also provides kits having chips containing the DNA of the biologically important polymorphisms for the elastin and fibrillin genes (and any of the other genes contributing to the functional integrity of elastin as noted above). Such chips permit the rapid detection of the polymorphisms, providing a convenient means for the rapid detection of those individuals at high or at low risk of developing restenosis or of atherosclerosis. The detection of specific polymorphisms in specific patients will allow highly specific and individualized treatment strategies to be devised for each patient to prevent or attenuate restenosis and or atherosclerosis.

The present invention, thus generally described, will be understood more readily by reference to the following example, which is provided by way of illustration and is not intended to be limiting of the present invention.

1. A method of assessing the risk of developing restenosis or atherosclerosis in an individual, comprising detecting in a sample obtained from said individual the presence of one or more biologically important polymorphisms in at least one gene selected from the group consisting of elastin, fibrillin, fibrillin-1, fibrillin-2, lysyl oxidase, microfibril-associated glycoprotein (MAGP), biglycan, osteopontin decorin.

2. (canceled)

3. The method according to claim 1, wherein an individual at high risk for the development of restenosis or atherosclerosis is indicated by the presence of one or more polymorphisms in said genes.

4. The method according to claim 1, wherein said sample comprises venous or arterial blood of said individual.

5. The method according to claim 1, wherein said sample comprises vascular tissue of said mammal.

6. The method according to claim 5, wherein said vascular tissue is vascular arterial tissue.

7. The method according to claim 1, wherein the presence or absence of said polymorphisms is detected using at least one genetic microarray.

8. The method according to claim 1, wherein the presence or absence of said polymorphisms is detected using PCR.

9. A method of assessing the risk of developing restenosis or athersclerosis in an a individual, comprising assaying in a sample obtained from said individual the level of expression of at least one gene selected from the group consisting of elastin, fibrillin, fibrillin-1, fibrillin-2, lysyl oxidase, microfibril-associated glycoprotein (MAGP), biglycan, osteopontin, and decorin.

10. (canceled)

11. The method according to claim 9, wherein the level of gene expression is determined by assaying the level of protein expression in a sample.

12. The method according to claim 9, wherein said sample is blood.

13. The method according to claim 9, wherein said sample is lymph.

14. The method according claim 9, wherein the level of protein expressions is determined by ELISA.

15. A method of inhibiting restenosis or atherosclerosis comprising administering to a patient at risk of developing restenosis or atherosclerosis a composition that inhibits smooth muscle cell proliferation or neointimal hyperplasia and wherein said composition modifies expression of at least one gene selected from the group consisting of elastin, fibrillin, fibrillin-1, fibrillin-2, lvsyl oxidase, microfibril-associated glycoprotein (MAGP), biglycan; osteopontin, and decorin.

16. (canceled)

17. The method according to claim 15, wherein the composition induces the expression of a gene or gene transcript that ameliorates the processes involved in restenosis or atherosclerosis.

18. The method according to claim 15, wherein said composition inhibits expression of one or more genes that promote smooth muscle cell proliferation or neointimal hyperplasia.

19. The method according to claim 15, wherein said composition comprises an antisense oligonucleotide.

20. The method according to claim 15, wherein said composition comprises an oligonucleotide that binds to mRNA to form a triplex.

21. The method according to claim 15, wherein said composition inhibits the activity of at least one protein selected from the group consisting of elastin, fibrillin, fibrillin-1, fibrillin-2, lysyl oxidase, microfibril-associated glycoprotein (MAGP), biglycan, osteopontin, and decorin.

22. The method according to claim 15, wherein said composition comprises an antibody that binds to a protein encoded by a gene selected from the group consisting of elastin, frbrillin, fibrillin-1, fibrillin-2, lysyl oxidase, microfibril-associated glycoprotein (MAGP), biglycan, osteopontin, and decorin.

23. The method according to claim 22, wherein said composition comprises a human antibody.

24. The method according to claim 15, wherein said composition comprises a soluble protein receptor.

25. The method according to claim 15, wherein said composition comprises a protein that is administered to supplement the loss of a Protein encoded by a gene selected from the group consisting of elastin, fibrillin, fibrillin-1, fibrillin-2, lysyl oxidase, microfibril-associated glycoprotein (MAGP), biglycan, osteopontin, and decorin.

26. The method according to claim 1, wherein detection is carried out using a kit suitable for detecting biologically significant polymorphisms of the elastin or fibrillin genes, any of the genes contributing to the functional integrity of elastin or the genes which encode lysyl oxidase, microfibril-associated glycoprotein (MAGP), biglycan, osteopontin, or decorin.

27. A kit for assessing the risk of developing restenosis or artherosclerosis in a individual, comprising a genetic microarray or other method suitable for detecting, in a sample obtained from said individual, the presence of one or more biologically important polymorphisms in at least one gene selected from the group consisting of elastin, fibrillin, fibrillin-1, fibrillin-2, lysyl oxidase, microfibril-associated glycoprotein (MAGP), biglycan, osteopontin, and decorin.
